(19) United States
(12) Patent Application Publication
(10) Pub. No.: US 2009/0088847 A1
(43) Pub. Date: Apr. 2, 2009

(54) SURGICAL INSTRUMENT SYSTEM

(76) Inventors: Manoj Krishna, Yarm (GB); Tai Friesem, Barwick (GB); Steven Brown, Parkland, FL (US)

Correspondence Address: CHRISTOPHER & WEISBERG, P.A., 200 EAST LAS OLAS BOULEVARD, SUITE 2040, FORT LAUDERDALE, FL, 33301, US

(21) Appl. No.: 11/865,172

(22) Filed: Oct. 1, 2007

(51) Int. Cl. A61F 2/44

(52) U.S. Cl. 623/17.11

(57) ABSTRACT

The present invention provides a surgical instrument system including a first instrument defining a track extending along a length thereof, a second instrument releasably engageable with a prosthesis and having an engagement structure slidably engageable with the track of the first instrument, a third instrument having a cutting element and an engagement structure slidably engageable with the track of the first instrument, and a fourth instrument having a prosthesis trial element sized and shaped substantially similar to the prosthesis, and including an engagement structure slidably engageable with the track of the first instrument.

### CROSS-REFERENCE TO RELATED APPLICATION

n/a

### STATEMENT REGARDING FEDERALLY SPONSORED RESEARCH OR DEVELOPMENT

n/a

### FIELD OF THE INVENTION

The present invention relates to instrumentation for performing spinal arthroplasty, and in particular, tools and methods of use thereof for the placement of an intervertebral implant.

### BACKGROUND OF THE INVENTION

Degenerative changes in the spine can cause the loss of normal structure and/or function. For example, the intervertebral disc is prone to degenerative changes associated with aging, disease, and injury. Over time the collagen structure of the intervertebral disc weakens and may become structurally unsound. Additionally, the water and proteoglycan content decreases, thereby narrowing the space between the adjacent vertebrae, which can result in nerve root compression and pain. These changes can lead to the disc's inability to handle mechanical stress.

One form of treatment available for degenerative disc disease is intervertebral disc replacement, which involves the surgical removal of a portion or all of an intervertebral disc followed by the implantation of a prosthetic device. A prosthetic device is typically placed between the two adjacent vertebrae to fill the space left by the removed disc and to restore the natural, healthy range of motion to the spinal segment.

Such surgical procedures can present the surgeon with several challenges, especially where the disc is severely degenerative. When the natural disc is removed, the adjacent vertebral bodies collapse upon each other thereby requiring the bodies to be separated to enable the desired placement of the prosthesis. However, separation or distraction of the vertebral bodies beyond a certain degree can result in further injury or damage to the vertebrae. Conversely, where the disc is severely degenerative, the narrow disc space and lack of elasticity between the vertebrae can hinder the surgeon's ability to separate the vertebrae to a height sufficient to enable placement of the prosthesis, and may further limit the ability of the surgeon to properly align and implant a prosthetic structure.

In view of the associated difficulties, specialized tools have been developed to facilitate the placement of disc prostheses between adjacent vertebral bodies of a patient's spine. Among the known tools for performing such procedures are spinal distractors, e.g. spreaders, and insertion devices. In general, a distractor may be placed between adjacent vertebrae, and then used to expand the space between two vertebrae. Once the space between the vertebral bodies is sufficient to enable placement of a prosthesis, the prosthetic device can then be inserted, either manually or with an insertion tool, into the space to hold the adjacent vertebrae apart.

While some tools for spinal arthroplasty may have some effectiveness in assisting surgeons with the placement of disc prosthesis, the use of such tools can prove cumbersome. For example, insertion of a spreader and/or prosthesis insertion tool into the limited disc space can cause fracture of a vertebra. Moreover, once inserted, the spreaders can cause over-distraction of the vertebral bodies, and may further hinder placement of the prosthesis. In the presence of degenerative disease or chronic changes where the disc space has become narrow, it can be difficult to maintain an adequate interbody height and, at the same time, insert and properly position the implant. Misalignment, placement of a prosthesis either too far anteriorly of insufficiently anteriorly can lead to pain, postural problems and/or limited mobility. Moreover, displacement of the implant could injure and/or otherwise damage surrounding tissue structure. As such, despite existing tools, there remains a need for a device to facilitate the safe and accurate insertion of a disc prosthesis between adjacent vertebral bodies.

### SUMMARY OF THE INVENTION

The present invention advantageously provides a surgical instrument system for implanting a prosthesis into a spinal segment via a posterior route. The system may have a first instrument including a first elongate body defining a first end and a second end, a first handle portion at the first end, and a first tip portion at the second end, where the first tip portion is positionable between a first vertebral body and a second vertebral body. The first elongate body may define a track extending along a length thereof. The system may also include a second instrument having a second elongate body defining a first end and a second end, a second handle portion at the first end, and a tip element at the second end, where the tip element is releasably engageable from the second elongate body. The second elongate body may also define an engagement structure slidably engageable with the track of the first instrument. In a particular embodiment, the first instrument may include a nerve retractor, and the tip element of the second instrument may include one or more cutting edges, for example, a first cutting edge and a second cutting edge disposed approximately 180 degrees from the first cutting edge. In addition, the tip element of the second instrument may include one or more prosthesis trial elements having portions sized and shaped substantially similar to the prostheses available for implantation.

The surgical instrument system may further include a third instrument having a third elongate body defining a first end and a second end, and a third handle portion at the first end. The second end of the third elongate body may be releasably engageable with the prosthesis, and the third elongate body may similarly define an engagement structure slidably engageable with the track of the first instrument. In addition, the surgical instrument system may include a fourth instrument including a fourth elongate body defining a first end and a second end, a fourth handle portion at the first end, and a fourth tip portion at the second end, wherein the fourth elongate body defines a track extending along a length thereof, and wherein the fourth instrument is releasably engageable with the first instrument.

A locking mechanism may be included on the first instrument for selectively controlling the engagement of a portion of the second and/or third instruments with the track. For example, the locking mechanism may include a protrusion proximate a portion of the track, where the protrusion is configurable from a retracted position such that engagement with the track is substantially uninhibited to an extended position such that engagement with the track is substantially inhibited. In addition, either and/or both of the second and third instruments may include an actuator mechanically coupled to the either the tip element (i.e., the cutting element and/or prosthesis trial element), such that the actuator facilitates the releasable engagement of the tip element to the respective elongate body.

An embodiment of the instrument system may also include a first instrument including a first elongate body defining a first end and a second end, a first handle portion at the first end, and a first tip portion at the second end, such that the first tip portion is positionable between a first vertebral body and a second vertebral body. The first elongate body may define a track extending along a length thereof. The system may include a second instrument having a second elongate body defining a first end and a second end, and a second handle portion at the first end, where the second end of the second elongate body is releasably engageable with the prosthesis. The second elongate body may also define an engagement structure slidably engageable with the track of the first instrument. A third instrument may be included having a third elongate body defining a first end and a second end, a third handle portion at the first end, and a cutting element at the second end. The cutting element may define a first cutting edge and a second cutting edge, and the third elongate body may define an engagement structure slidably engageable with the track of the first instrument. Alternatively, the third instrument may include a prosthesis trial element at the second end, where the prosthesis trial element includes a portion sized and shaped substantially similar to the prosthesis. The third instrument may include an actuator mechanically coupled to the cutting element, such that the actuator is able to facilitate the releasable engagement of the cutting element to the third elongate body.

A method of using the surgical instrument system for the present invention may entail the steps of positioning a first tool having a substantially flattened tip portion between a first vertebral body and a second vertebral body; slidably engaging a second tool to the first tool, where the second tool includes a tip element releasably engageable thereto; and positioning the tip element between the first vertebral body and the second vertebral body. The method may also include the use of a cutting element having one or more cutting edges, such that the step of positioning the tip element between the first vertebral body and the second vertebral body includes making a cut in at least one of the first and second vertebral bodies. In addition to and/or in the alternative, the tip element may include a prosthesis trial element having a portion sized and shaped substantially similar to the prosthesis. The steps of disengaging the second tool from the first tool; slidably engaging a third tool to the first tool, where the third tool is releasably engaged to the prosthesis; and positioning the prosthesis between the first vertebral body and the second vertebral body may also be performed.

Moreover, a method of using the surgical system may include the steps of positioning a fourth tool at least partially between the first vertebral body and the second vertebral body and releasably engaging the fourth tool with the first tool such that the fourth tool and the first tool are substantially parallel. The method may further include disengaging the second or third tool from the first tool and slidably engaging the second or third tool to the fourth tool such that the tip element is positioned between the first vertebral body and the second vertebral body.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is an illustration of an embodiment of a surgical instrument system in accordance with the present invention;

FIG. 2 is an illustration of an embodiment of a guide tool in accordance with the present invention;

FIG. 3 is an additional illustration of an embodiment of a guide tool in accordance with the present invention;

FIG. 4 is an illustration of an embodiment of a cutting tool in accordance with the present invention;

FIG. 5 is an additional illustration of an embodiment of a cutting tool in accordance with the present invention;

FIG. 6 is an assembly illustration of an engagement between an embodiment of a cutting tool coupled to a guide tool in accordance with the present invention;

FIG. 7 is an illustration of an embodiment of a prosthesis trial tool in accordance with the present invention;

FIG. 8 is an additional illustration of an embodiment of a prosthesis trial tool in accordance with the present invention;

FIG. 9 is an assembly illustration of an engagement between an embodiment of a prosthesis trial tool and a guide tool in accordance with the present invention;

FIG. 10 is an illustration of an embodiment of a prosthesis insertion tool in accordance with the present invention;

FIG. 11 is an additional illustration of an embodiment of a prosthesis insertion tool in accordance with the present invention;

FIG. 12 is an illustration of an embodiment of a prosthesis insertion tool coupled to a guide tool in accordance with the present invention;

FIG. 13 is an additional illustration of an embodiment of a prosthesis insertion tool coupled to a guide tool in accordance with the present invention;

FIG. 14 is an illustration of a portion of a spinal column;

FIG. 15 is a side view of an embodiment of a guide tool of the present invention positioned within a portion of a spinal column;

FIG. 16 is a top view illustration of an embodiment of a guide tool of the present invention positioned within a portion of a spinal column;

FIG. 17 is an additional top view illustration of an embodiment of a guide tool coupled to an additional tool of the present invention and positioned within a portion of a spinal column;

FIG. 18 is a perspective view of a plurality of guide tools engaged with a portion of a spinal column; and

FIG. 19 is an additional perspective view of a plurality of guide tools engaged with a portion of a spinal column.

### DETAILED DESCRIPTION OF THE INVENTION

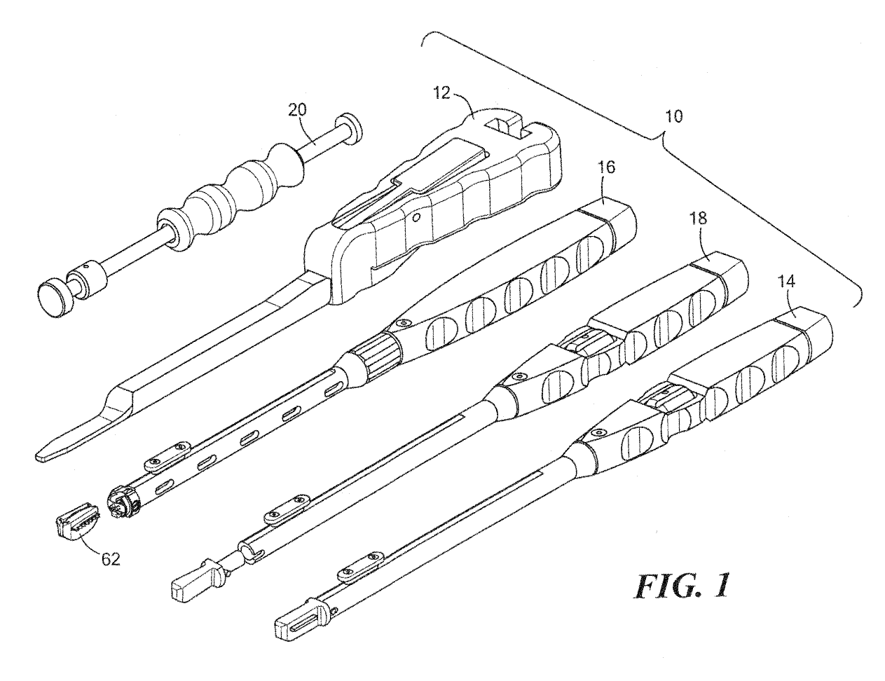

The present invention provides an instrument system for spinal arthroplasty including one or more tools to aid in the preparation, positioning, placement, and/or manipulation of a portion of a spinal column, as well as an implant or prosthesis intended for implantation into the spinal column. Now referring to FIG. 1, the instrument system 10 of the present invention may generally include a guide tool 12, a cutting tool 14, a prosthesis insertion tool 16, and/or a prosthesis trial tool 18. The system 10 may further include a force application tool 20, such as a slap hammer or the like, to facilitate placement and/or insertion of one or more tools of the instrument system 10. The tools may be movably engageable with one another to perform a particular task during a surgical procedure, as described in more detail below.

The guide tool 12 of the present invention may be positionable and/or engageable with a portion of a spinal column. For example, the guide tool 12 may be positionable between first and second vertebral bodies, and may further be positioned such that at least a portion of the guide tool 12 is insertable into a portion of an intervertebral space of region between first and second vertebral bodies to distract or otherwise manipulate the first and second vertebral bodies into a desired orientation. Once in a desired position, the guide tool 12 may support the first and second vertebral bodies to maintain a space or implantation site therebetween.

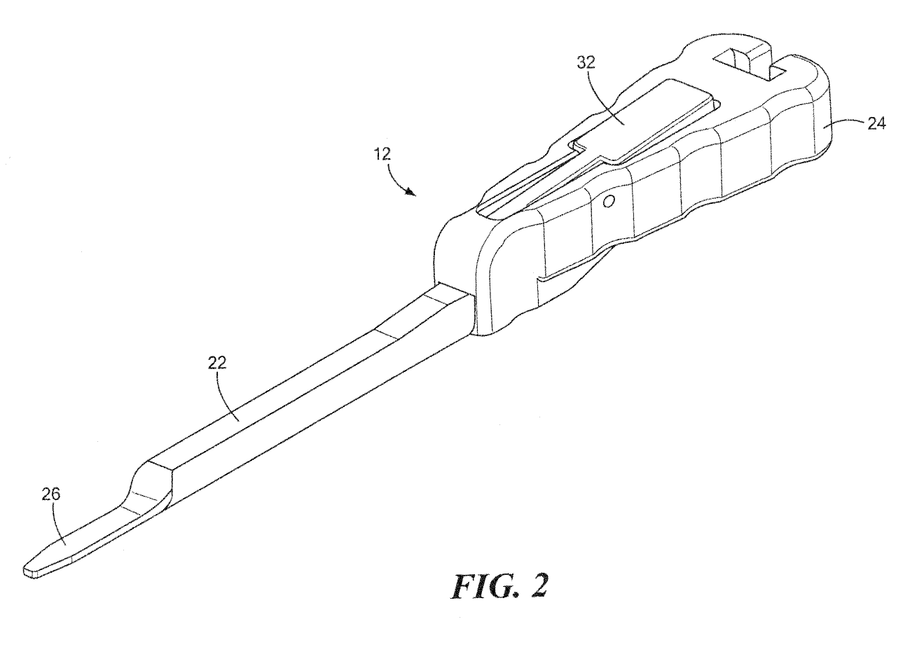

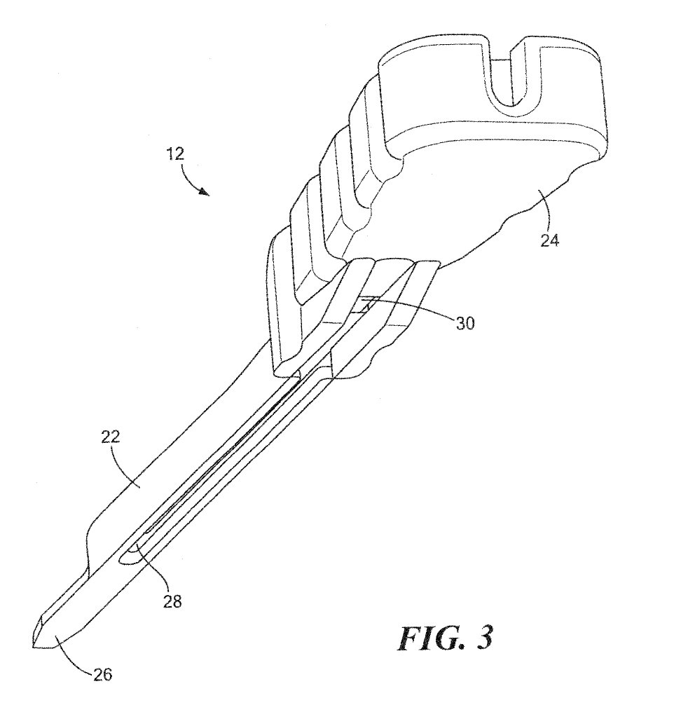

Now referring to FIGS. 2 and 3, in a particular embodiment, the guide tool 12 may include a nerve retractor defining an elongated body 22 having a handle portion 24 at a first end, and an insertion portion 26 at a second end opposite to the first end. The elongated body 22 may be substantially cylindrical with one or more flattened surfaces, and/or may include a hollow portion therein. The handle portion 24 may have a flared shape (or any other ergonomically practicable shape) to provide additional graspable surface area for ease of use, where the handle may define a depression or shaped surface that is releasably engageable with a slap hammer or similar tool. The insertion portion 26 of the guide tool 12 may have a tapered or narrowed shape for placement into compressed or constricted region of tissue. In particular, the insertion portion 26 may include a substantially flattened, blade-like planar tip extending from the cylindrical elongate body of the guide tool 12. The elongated body 22 of the guide tool 12 may further define a track 28, where the track 28 may include a hollowed or carved out groove depressed into or otherwise extending along the elongated body 22. The track 28 may extend along a significant portion of the length of the guide tool 12 between the handle portion and the insertion portion, and may generally define a hollowed “T” shaped cross section. The track 28 may also include an enlarged opening at an end of the track 28 in proximity to the handle portion of the elongated body 22 of the guide tool 12. When viewed from the side the enlarged opening may narrow along the length of the elongated body 22 to form the bottom portion of the “T” shaped cross section of the track 28.

The guide tool 12 may further include a locking mechanism proximate a portion of the track 28 and/or the handle portion to prevent and/or allow engagement of a portion of a tool or device with the track 28. For example, the locking mechanism may include a movable protrusion 30 near an opening or end of the track 28 to prevent and/or allow a portion of a tool or device to enter and/or align with the track 28. In particular, the locking mechanism may include a button that protrudes from an underside of the handle adjacent to the track 28 opening. The button may be coupled to a lever 32 or similar movable construct to either controllably extend or retract the button. In addition, the locking mechanism may be biased to maintain a particular configuration, i.e., to have the button either in an extended or retracted position, which may be accomplished by a spring or other biasing element coupled to the button, lever, and/or other component of the locking mechanism.

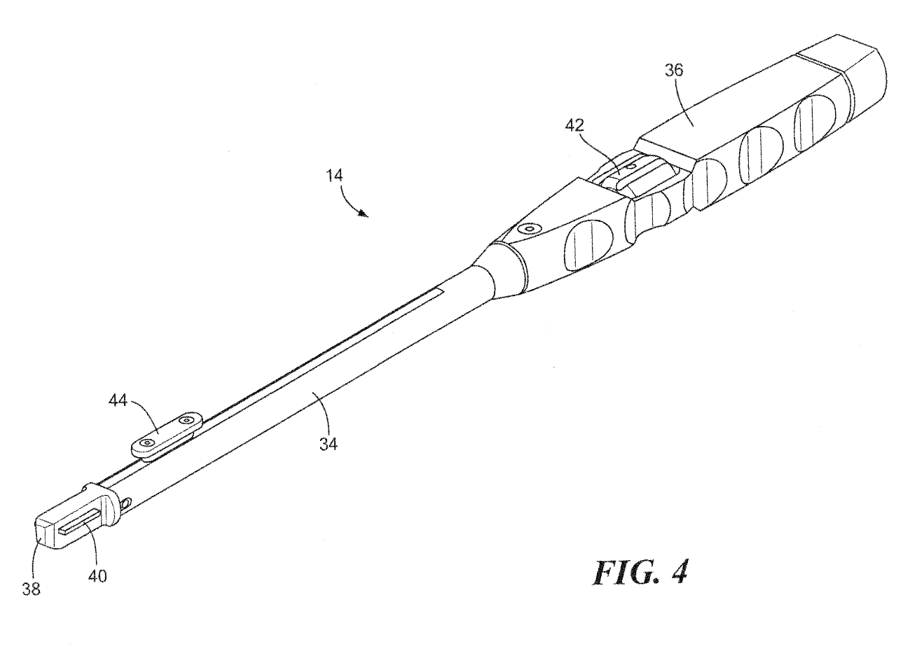

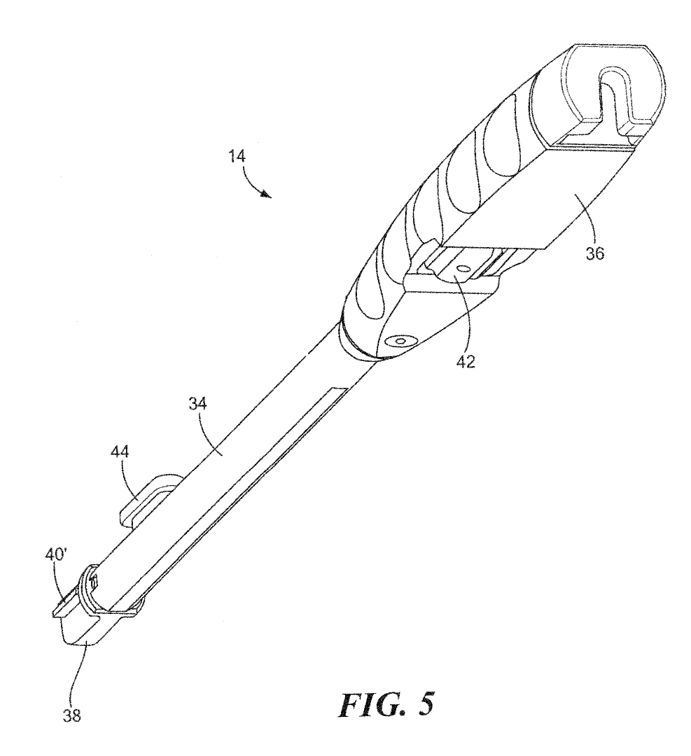

The system 10 of the present invention may also include the cutting tool 14, which may be engageable with or otherwise movable about the guide tool 12. As shown in FIGS. 4 and 5, the cutting tool 14 may generally define an elongated body 34 having a handle portion 36 at a first end, and a cutting element 38 disposed at a second end opposite to the first end. Similar to the guide tool 12, the handle portion 36 of the cutting tool 14 may have a flared shape (or any other ergonomically practicable shape) to provide additional graspable surface area for ease of use. The cutting element 38 may be removable from the elongate body 34 of the tool, and may include one or more sharpened edges positionable about a tissue region, such as an intervertebral disc or the like. The cutting element 38 may be sized and shaped such that it is positionable between first and second vertebral bodies, and further, may include a contoured shape that closely approximates or mimics the shape and dimensions of a prosthesis to be implanted. In addition, the cutting element 38 may include one or more cutting edges positioned about the cutting element body corresponding to one or more projections, or anchoring features of a particular implantable prosthesis. For example, the cutting element may include a first cutting edge 40 and a second cutting edge 40′ extending from the cutting element 38 where the first and second cutting edges may be positioned directly opposite one another, i.e., oriented 180 degrees from one another in a substantially parallel configuration.

The cutting tool 14 may also include a cutting element retention mechanism for selectively coupling and releasing the cutting element 38 to and from the elongate body 34. For example, a retention actuator 42 may be coupled to or otherwise disposed in proximity to the handle 36 of the cutting tool 14. The retention actuator 42 may be coupled to the second end of the elongate body 34 through a linkage mechanism (not shown) that interacts with and/or is able to releasably engage the cutting element 38. In operation, manipulation of the retention actuator 42 may cause the cutting element 38 to be securely affixed to the second end of the elongate body, and may further facilitate the removal of the cutting element 38 from the second end of the elongate body. The retention actuator 42 may include a button, lever, thumb wheel and/or similar controllable mechanical mechanism or actuator as known in the art.

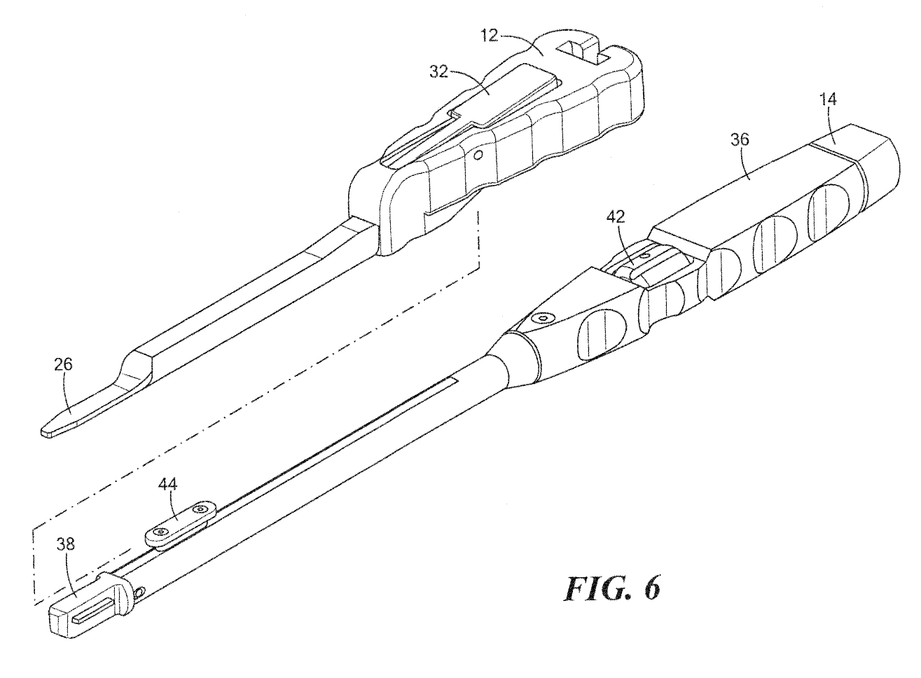

The cutting tool 14 may further include an engagement structure 44 that is matable with and/or otherwise complementary to the track 28 of the guide tool 12, such that the cutting tool 14 is able to movably couple to the guide tool 12, as shown in FIG. 6. For example, the cutting tool 14 may include a “T” shaped protrusion or other structure that is slidably receivable by the track 28 of the guide tool 12. The engagement structure 44 of the cutting tool 14 may be situated along a length of the elongated body 34, and may further be positioned near the second end of the cutting tool 14 in proximity to the cutting element 38.

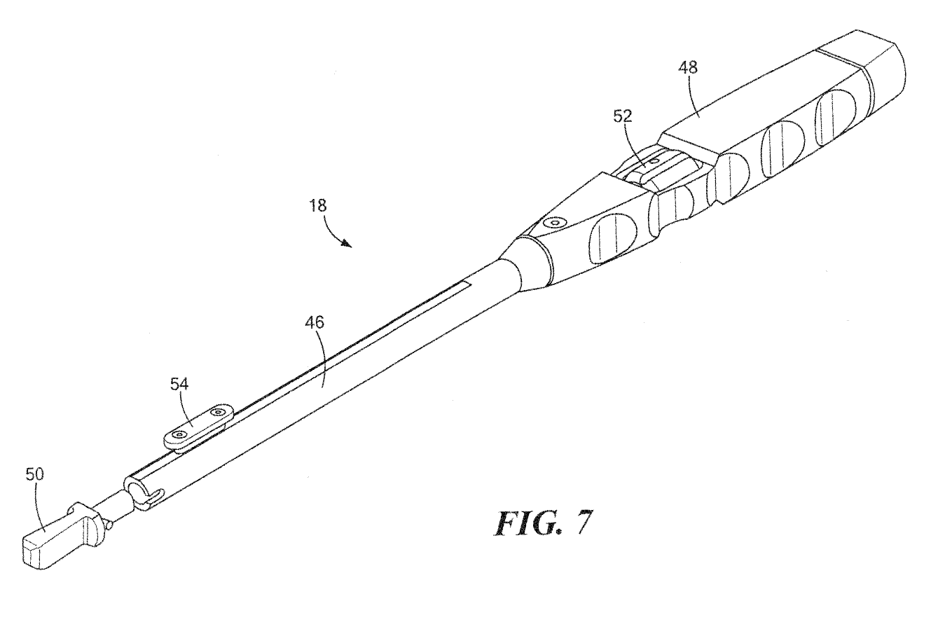

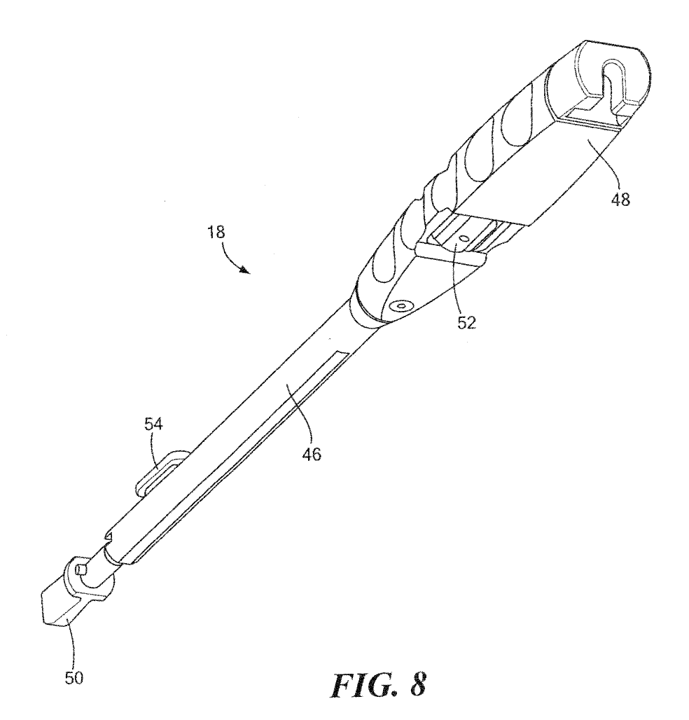

The instrument system 10 of the present invention may also include the prosthesis trial tool 18 to aid in the selection and/or sizing of a prosthesis to be implanted into a surgical site. As shown in FIGS. 7 and 8, the prosthesis trial tool 18 may generally define an elongated body 46 having a handle portion 48 at a first end, and a prosthesis trial element 50 disposed at a second end opposite to the first end. Similar to both the guide tool 12 and the cutting tool 14, the handle portion of the prosthesis trial tool 18 may have a flared shape (or any other ergonomically practicable shape) to provide additional graspable surface area for ease of use. The prosthesis trial element 50 may be removable from the elongate body of the tool, and may be sized and shaped such that it is positionable between first and second vertebral bodies. The prosthesis trial element 50 may include a contoured shape that closely approximates or mimics the shape and dimensions of a prosthesis to be implanted. In addition, a plurality of prosthesis trial elements may be included, with the trial elements having varying dimensions correlating to available prosthesis dimensions to aid in determining an appropriate prosthesis for implantation in a particular situation.

The prosthesis trial tool 18 may also include a trial element retention mechanism for selectively coupling and releasing the trial element to and from the elongate body. The trial element retention mechanism may operate substantially similar to that of the cutting element retention mechanism described above. For example, a retention actuator 52 may be coupled to or otherwise disposed in proximity to the handle of the prosthesis trial tool 18. The retention actuator 52 may be coupled to the second end of the elongate body 46 through a linkage mechanism (not shown) that interacts with and/or is able to releasably engage the prosthesis trial element. In operation, manipulation of the retention actuator 52 may cause the prosthesis trial element 50 to be securely affixed to the second end of the elongate body, and may further facilitate the removal of the prosthesis trial element 50 from the second end of the elongate body 46. The retention actuator 52 may include a button, lever, thumb wheel and/or similar mechanical mechanism or actuator as known in the art.

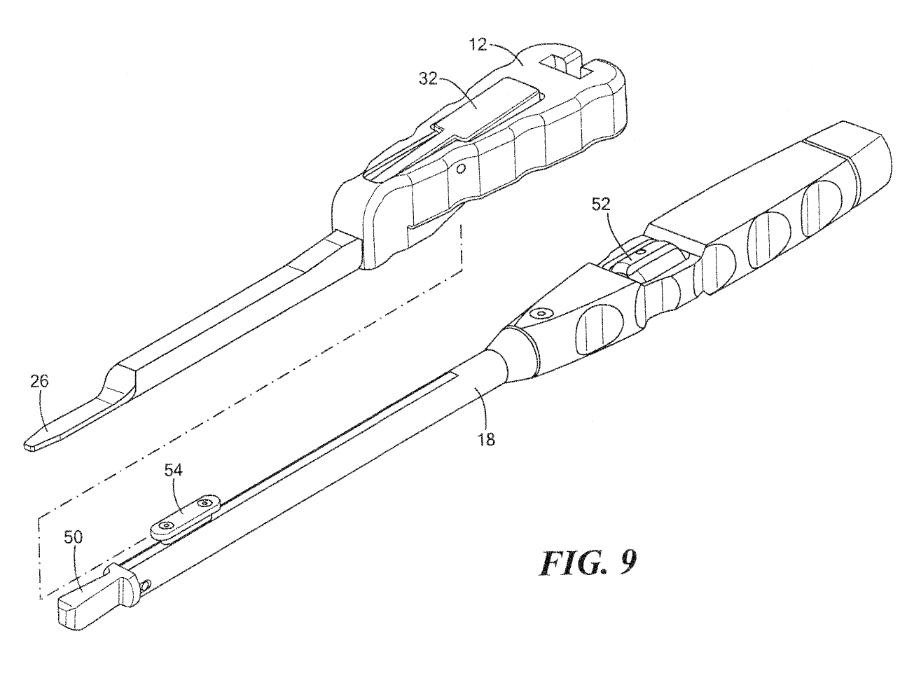

Similar to the cutting tool 14, the prosthesis trial tool 18 may further include an engagement structure 54 that is matable with and/or otherwise complementary to the track 28 of the guide tool 12, such that the prosthesis trial tool 18 is able to movably couple to the guide tool 12 as shown in FIG. 9. For example, the prosthesis trial tool 18 may include a “T” shaped protrusion or other structure that is slidably receivable by the track 28 of the guide tool 12. The engagement structure 54 of the prosthesis trial tool 18 may be situated along a length of the elongated body, and may further be positioned near the second end of the prosthesis trial tool 18 in proximity to the trial element.

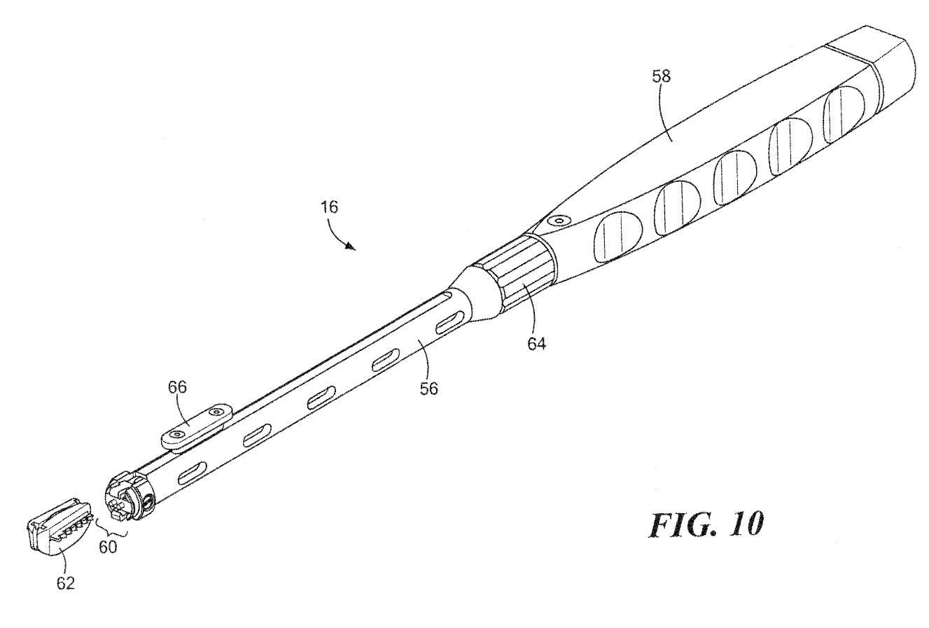

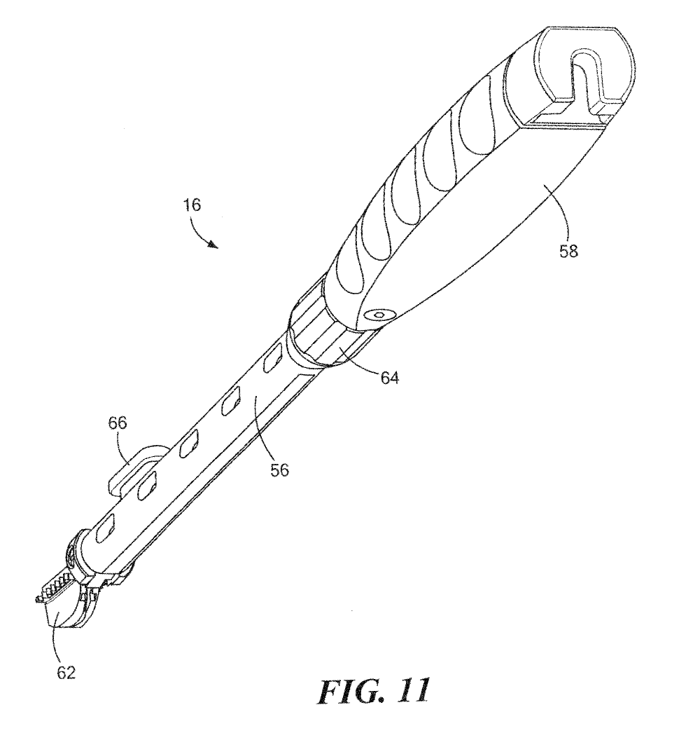

The instrument system 10 of the present invention may also include the prosthesis insertion tool 16 to aid in positioning a prosthesis into a surgical site. As shown in FIGS. 10 and 11, the prosthesis insertion tool 16 may generally define an elongated body 56 having a handle portion 58 at a first end, and a prosthesis engagement mechanism 60 disposed at a second end opposite to the first end, where the prosthesis engagement mechanism 60 provides for the selective engagement of a prosthetic device 62. Similar to both the guide tool 12 and the cutting tool 14, the handle portion of the prosthesis trial tool 18 may have a flared shape (or any other ergonomically practicable shape) to provide additional graspable surface area for ease of use.

The prosthesis engagement mechanism 60 of the prosthesis insertion tool 16 may operate substantially similar to that of the cutting element retention mechanism described above. For example, an engagement actuator 64 may be coupled to or otherwise disposed in proximity to the handle 58 of the prosthesis trial tool 18. The engagement actuator 64 may be coupled to the second end of the elongate body through a linkage mechanism (not shown) that interacts with and/or is able to releasably engage the prosthesis 62. In operation, manipulation of the retention actuator 64 may cause the prosthesis 62 to be securely affixed to the second end of the elongate body, and may further facilitate the separation of the prosthesis 62 from the second end of the elongate body 56. The retention actuator 64 may include a button, lever, thumb wheel and/or similar mechanical mechanism or actuator as known in the art.

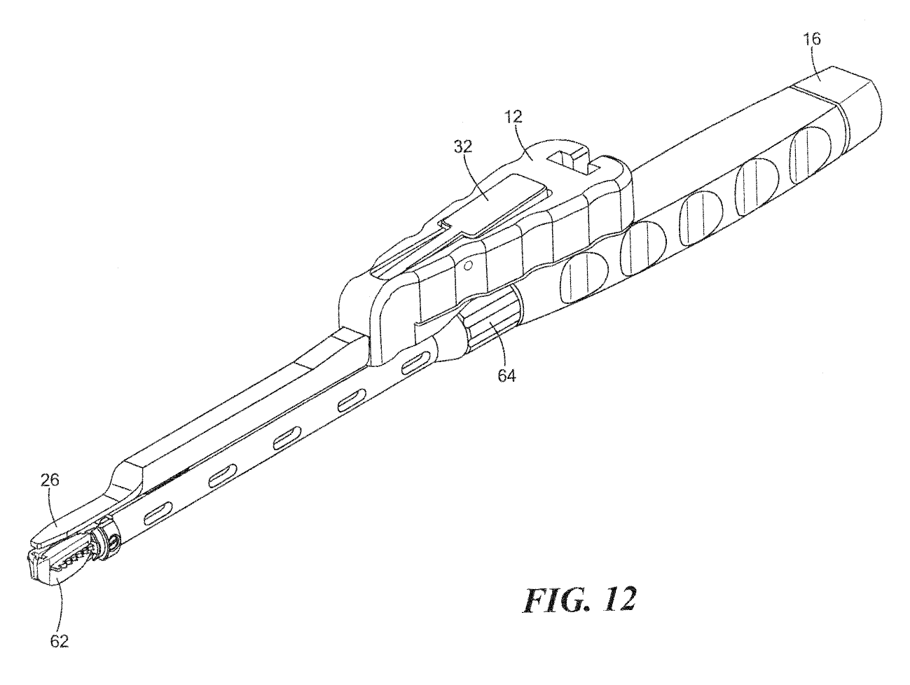

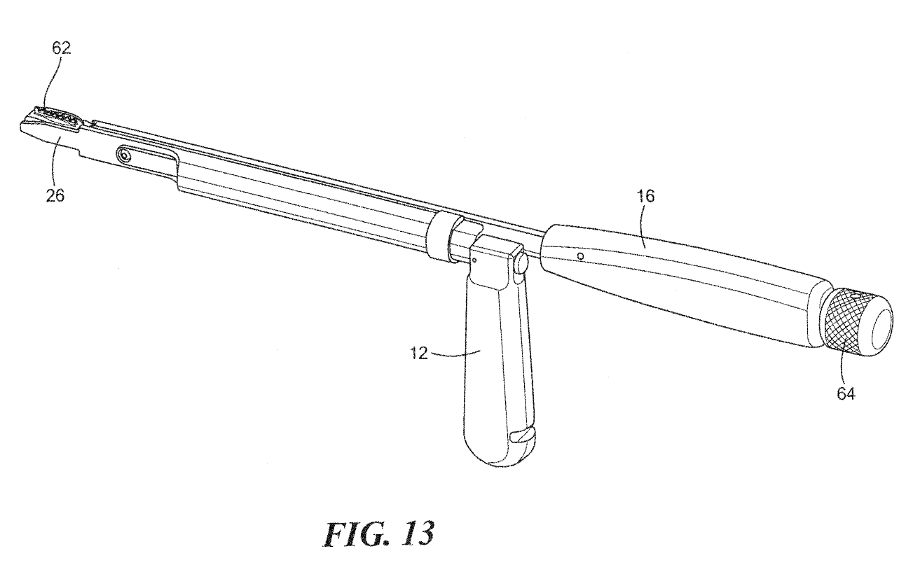

The prosthesis insertion tool 16 may further include an engagement structure 66 that is matable with and/or otherwise complementary to the track 28 of the guide tool 12, such that the prosthesis insertion tool 16 is able to movably couple to the guide tool 12 as shown in FIGS. 12 and 13. For example, the prosthesis insertion tool 16 may include a “T” shaped protrusion or other structure that is slidably receivable by the track 28 of the guide tool 12. The engagement structure 66 of the prosthesis insertion tool 16 may be situated along a length of the elongated body, and may further be positioned near the second end of the prosthesis trial tool 18 in proximity to the cutting element.

Of note, although the tools above have been describes and illustrated as individual and distinct tools, the features, components, and characteristics of the above-described devices may be included in a single tool or a reduced number of tools. For example, an embodiment of the instrument system 10 of the present invention may include the guide tool 12, and an additional tool that is slidably engageable with the guide tool 12. The tool may include an elongate body, a handle and the like similar to those tools described above, and may further include a retention mechanism able to releasably couple to one or more of the cutting elements, prosthesis trial elements, and/or prostheses.

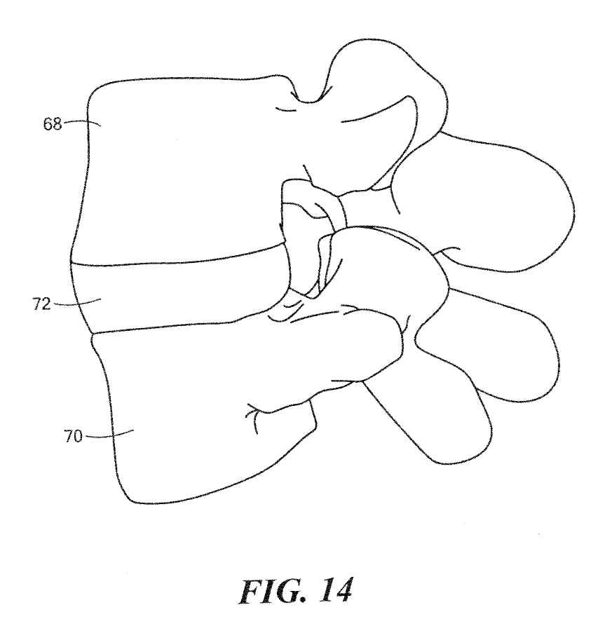

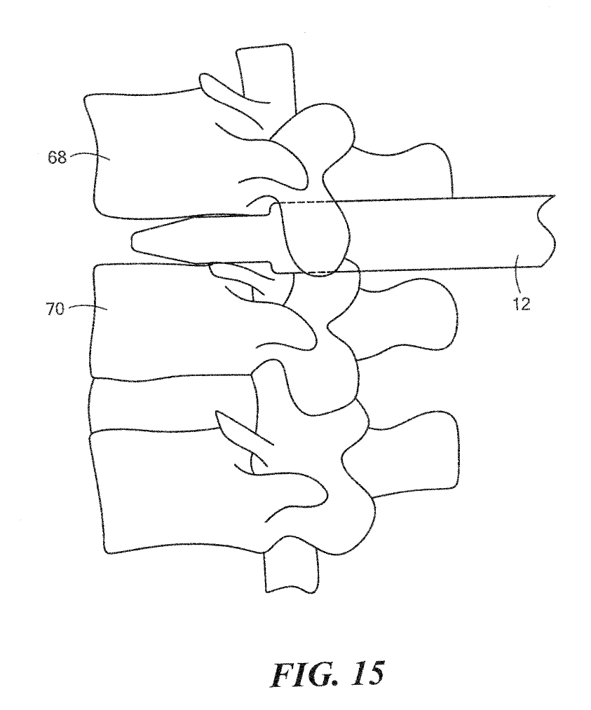

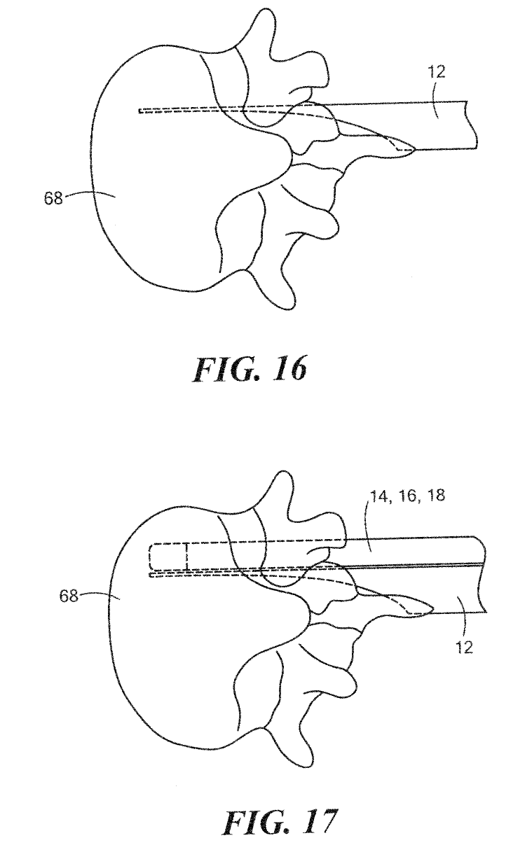

In an exemplary use, the instrument system 10 of the present invention may be employed in a spinal arthroplasty or fusion procedure involving the placement of an intervertebral disc or cage prosthesis into a portion of a spinal segment. Referring to FIG. 14, a spinal segment generally includes a first vertebral body 68, a second vertebral body 70, and an intervertebral disc 72. The spinal prosthesis may include any number of available intervertebral disc prostheses, including but not limited to spinal cages, fusion devices, articulating prostheses, and other devices known in the art. In a particular example, the spinal prosthesis may include a paired-disc device having an articulating range of motion, where each of the disc components include an anchoring element or protruding part to engage a vertebral body both above and below the implantation site. Primarily, one or more incisions may be made to expose a particular surgical site, i.e., a portion of the spinal column or a spinal segment in need of repair. A diseased and/or damages intervertebral disc may be removed in anticipation of the implantation of a spinal prosthesis. Now referring to FIGS. 15 and 16, upon appropriate preparation of the surgical site, the insertion portion of the guide tool 12 may be positioned between the first and second vertebral bodies of the spinal segment. When seated between vertebral bodies, the flattened, substantially planar tip of the guide tool 12 may retract and protect the nerve root and spread or otherwise maintain a spacing between the vertebral bodies.

Now referring to FIG. 17, subsequent to achieving the desired positioning of the guide tool 12, the prosthesis trial tool 18 may be coupled to the guide tool 12 through the slidable engagement of the engagement structure of the prosthetic trial tool 18 with the track 28 of the guide tool 12. The coupling of the two tools may be facilitated by the locking mechanism of the guide tool 12. For example, the movable protrusion proximate the track 28 of the guide tool 12 can be retracted to allow the engagement structure to couple to the track 28, with the movable protrusion subsequently extending to secure the positioning of the engagement structure in the track 28 and to further prevent the unintentional decoupling of the two tools.

As described above, the prosthesis trial tool 18 may include a prosthesis trial element 50 having a portion sized and shaped to closely approximate the size and shape of a prosthesis available for implantation. Moreover, a plurality of trial elements may be available having varying shapes and sizes to aid in the selection of an appropriately sized and shape prosthesis for a particular patient. Once the prosthesis trial tool 18 has been coupled to the guide tool 12, the prosthesis trial tool 18 may be slidable moved along the guide tool 12 to position a trial element 50 between the first and second vertebral bodies of the surgical site to determine which size and/or shape prosthesis would provide a desirable fit. Of course, should an initial prosthesis trial element 50 fail to provide a desired fit or indication of success, subsequent trial element having varying dimensions may be releasably engaged to the prosthesis trial tool 18 until a desired fit has been identified. The retention mechanism of the prosthesis trial tool 18 may be manipulated to facilitate the coupling and releasing of one or more prosthesis trial elements from the tool body during this portion of the procedure.

Upon achieving a desired fit of a specific prosthesis trial element, a correlating prosthesis may be selected for actual implantation. The prosthesis trial tool 18 may be removed from the surgical site and disengaged with the track 28 of the guide tool 12. The cutting tool 14 may then be coupled to the guide tool 12 in similar fashion to that of the prosthesis trial tool 18, i.e., the engagement structure 44 may be slidably engaged with the track 28, where the engagement may be facilitated by the locking mechanism. The cutting tool 14 may include the cutting element 38 having one or more cutting edges corresponding to the fins of the prosthesis. The cutting tool 14 may be slidably positioned along the length of the guide tool 12 to place the cutting element 38 between the first and second vertebral bodies such that the one or more cutting edges create an incision, cut, or similar mark in the first and second vertebral bodies in anticipation of the placement of the fins of the prosthesis. A particular cutting element 38 used to create the incisions may be selected from a plurality of cutting elements having dimensions correlating to available prostheses, with the desired cutting element being removably engaged with the cutting tool 14 upon a determination of the prosthesis to be used.

After making the desired incisions, the cutting tool 14 may be disengaged from the guide tool 12, and the prosthesis insertion tool 16 may then be slidably engaged to the guide tool 12 as described above. The prosthesis insertion tool 16 may have the prosthesis 62 to be implanted releasably coupled to the end of the tool, i.e., the prosthesis engagement mechanism 60 may mate with a feature on the prosthesis such as a groove, lip, depression, etc. The prosthesis insertion tool 16 may then be slidable positioned along the length of the guide tool 12 to place the prosthesis 62 between the first and second vertebral bodies. As the guide tool 12 has provided a static anchor to which the previous tools have been engaged, the prosthesis should align directly with the prepared space and any incisions therein provided by the cutting tool 14. Once the prosthesis 62 is properly positioned, the engagement mechanism 60 of the insertion tool 16 may be actuated to release the prosthesis form the end of the tool. The prosthesis insertion tool 16 may then be retracted and disengaged from the guide tool 12, and the guide tool 12 may subsequently be removed from the surgical site as well. For a paired-disc prosthesis, the guide tool 12 may be rotated and the above steps may be repeated for insertion of the complementary component of the prosthesis on the opposite side of the surgical site.

By initially placing the guide tool 12 in a desired position, it provides a secure and reliable guide for subsequent tools needed to prepare and/or otherwise perform the steps of a surgical procedure. As such, the likelihood that the preparation of a surgical site and the subsequent positing of an implant are misaligned, offset, or otherwise inaccurate is greatly reduced.

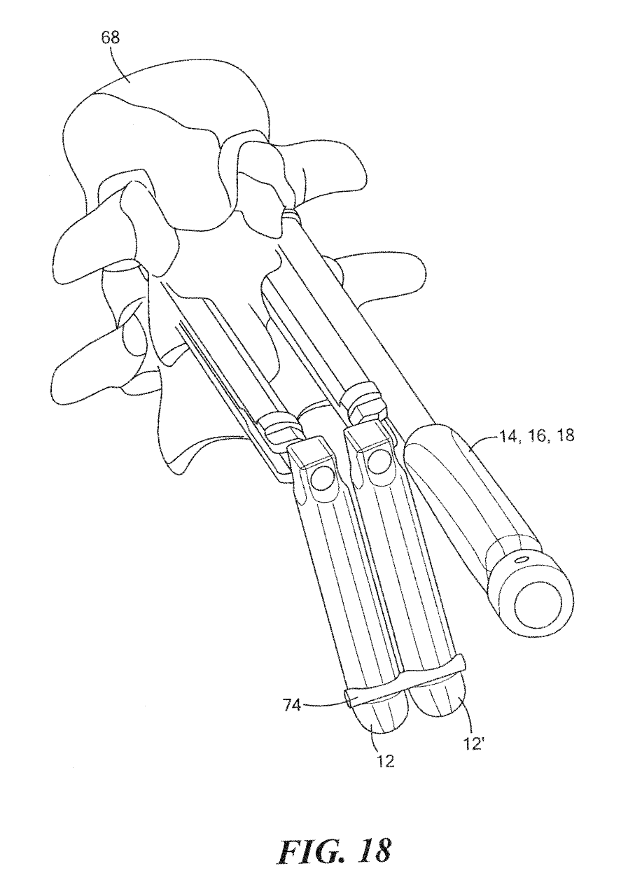

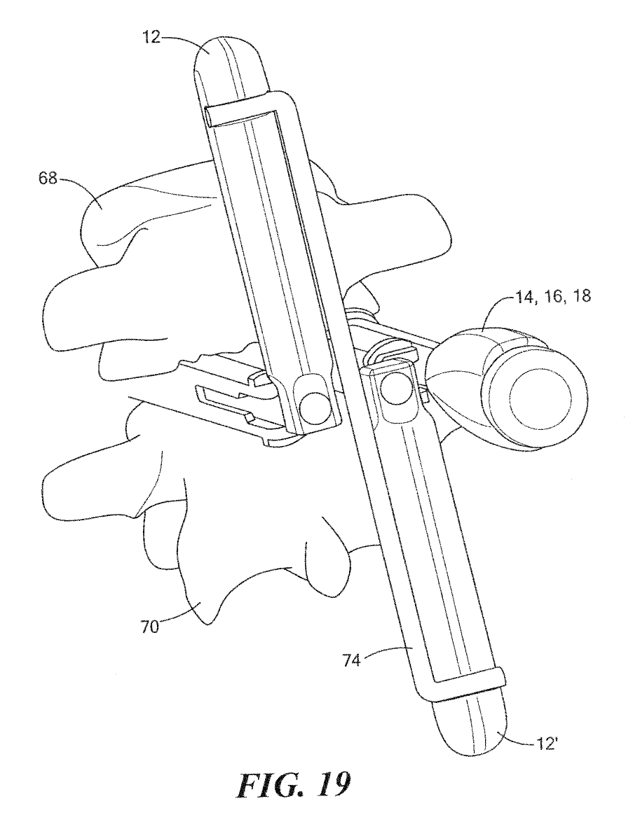

Now referring to FIGS. 18 and 19, an exemplary use of the surgical instrument system of the present invention may include the use of a plurality of guide tools for the preparation of a surgical site and subsequent placement of one or more prostheses. For example, first and second guide tools 12, 12′ may be positioned within an intervertebral space, similar to that described above. The first and second guide tools 12, 12′ may be releasably engaged to one another through a connector 74 to ensure that the handles and/or elongate bodies of the first and second guide tools are substantially parallel. The connector 74 may include a rod, a latch, or other releasable engaging mechanisms as known in the art. The first and second guide tools may be connected in configuration where the handles are similarly oriented next to each other, as shown in FIG. 18, or with the handle portions inverted and a “Z”-shaped connector engaging the handles, as shown in FIG. 19. Either way, the elongate bodies, and thus the tracks, of the first and second guide tools 12, 12′ are substantially parallel and may receive any of the cutting tool, prosthesis trial tool, and/or the prosthesis insertion tool during a procedure for implantation.

By engaging the first and second guide tools 12, 12′ in a substantially parallel configuration, a plurality of prostheses may be implanted into the intervertebral site along parallel axes to ensure a matching orientation and/or position of the prostheses. In particular, where a matched pair of intervertebral prostheses is to be implanted into a single intervertebral space, the discs should be aligned as closely as possible to provide complementary movement and/or motion characteristics throughout their use. As such, by inserting the prostheses using the tools engaged with the first and second guide tools 12, 12′, the resulting matched positioning and orientation of the prostheses will reduce the likelihood that the prostheses are misaligned, and further ensure the synergistic and complementary movement characteristics of the prostheses and the spinal segment.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A surgical instrument system for implanting a prosthesis, comprising:
a first instrument including a first elongate body defining a first end and a second end, a first handle portion at the first end, and a first tip portion at the second end, and wherein the first elongate body defines a track extending along a length thereof; and
a second instrument having a second elongate body defining a first end and a second end, a second handle portion at the first end, and a tip element at the second end, wherein the second elongate body defines an engagement structure slidably engageable with the track of the first instrument.

2. The surgical instrument system according to claim 1, wherein the first tip portion has a substantially flattened, blade-like shape.

3. The surgical instrument system according to claim 1, wherein the first instrument is a nerve retractor.

4. The surgical instrument system according to claim 1, wherein the tip element of the second instrument includes one or more cutting edges.

5. The surgical instrument system according to claim 4, wherein the tip element includes a first cutting edge and a second cutting edge disposed approximately 180 degrees from the first cutting edge.

6. The surgical instrument system according to claim 1, wherein the tip element of the second instrument is a prosthesis trial element having a portion sized and shaped substantially similar to the prosthesis.

7. The surgical instrument system according to claim 1, wherein the tip element is releasably engageable from the second elongate body.

8. The surgical instrument system according to claim 7, further comprising a plurality of tip elements releasably engageable with the second elongate body, wherein the plurality of tip elements include a plurality of prosthesis trial elements having portions sized and shaped substantially similar to a plurality of prostheses.

9. The surgical instrument system according to claim 7, wherein the second instrument includes an actuator mechanically coupled to the tip element, wherein the actuator facilitates the releasable engagement of the tip element to the second elongate body.

10. The surgical instrument system according to claim 1, further comprising a third instrument having a third elongate body defining a first end and a second end, and a third handle portion at the first end, wherein the second end of the third elongate body is releasably engageable with the prosthesis, and wherein the third elongate body defines an engagement structure slidably engageable with the track of the first instrument.

11. The surgical instrument system according to claim 1, wherein the first instrument further includes a locking mechanism for selectively controlling the engagement of a portion of the second instrument with the track.

12. The surgical instrument system according to claim 11, wherein the locking mechanism includes a protrusion proximate a portion of the track, and wherein the protrusion is configurable from a retracted position such that engagement with the track is substantially uninhibited to an extended position such that engagement with the track is substantially inhibited.

13. The surgical instrument system according to claim 1, further comprising a force application tool engageable with at least one of the first and second handle portions.

14. The surgical instrument system according to claim 1, further comprising a third instrument including a third elongate body defining a first end and a second end, a third handle portion at the first end, and a third tip portion at the second end, wherein the third elongate body defines a track extending along a length thereof, and wherein the third instrument is releasably engageable with the first instrument.

15. A surgical instrument system for implanting a prosthesis, comprising:
a first instrument including a first elongate body defining a first end and a second end, a first handle portion at the first end, and a first tip portion at the second end, wherein the first elongate body defines a track extending along a length thereof; and
a second instrument having a second elongate body defining a first end and a second end, and a second handle portion at the first end, wherein the second end of the second elongate body is releasably engageable with the prosthesis, and wherein the second elongate body defines an engagement structure slidably engageable with the track of the first instrument.

16. The surgical instrument system according to claim 15, further comprising a third instrument having a third elongate body defining a first end and a second end, a third handle portion at the first end, and a cutting element at the second end, wherein the cutting element defines a first cutting edge and a second cutting edge, wherein the third elongate body defines an engagement structure slidably engageable with the track of the first instrument.

17. The surgical instrument system according to claim 16, wherein the third instrument includes an actuator mechanically coupled to the cutting element, wherein the actuator facilitates the releasable engagement of the cutting element to the third elongate body.

18. The surgical instrument system according to claim 15, further comprising a third instrument having a third elongate body defining a first end and a second end, a third handle portion at the first end, and a prosthesis trial element at the second end, wherein the prosthesis trial element includes a portion sized and shaped substantially similar to the prosthesis, and wherein the third elongate body defines an engagement structure slidably engageable with the track of the first instrument.

19. The surgical instrument system according to claim 18, wherein the third instrument includes an actuator mechanically coupled to the prosthesis trial element, wherein the actuator facilitates the releasable engagement of the prosthesis trial element to the third elongate body.

20. The surgical instrument system according to claim 15, wherein the first instrument further includes a locking mechanism for selectively controlling the engagement of a portion of the second instrument with the track.

21. The surgical instrument system according to claim 20, wherein the locking mechanism includes a protrusion proximate a portion of the track, and wherein the protrusion is configurable from a retracted position such that engagement with the track is substantially uninhibited to an extended position such that engagement with the track is substantially inhibited.

22. The surgical instrument system according to claim 15, further comprising a third instrument including a third elongate body defining a first end and a second end, a third handle portion at the first end, and a third tip portion at the second end, wherein the third elongate body defines a track extending along a length thereof, and wherein the third instrument is releasably engageable with the first instrument.

23. A surgical instrument system for implanting a prosthesis, comprising:
a first instrument including a first elongate body defining a first end and a second end, a first handle portion at the first end, and a first tip portion at the second end, wherein the first tip portion has a substantially flattened, blade-like shape, wherein the first elongate body defines a track extending along a length thereof;
a second instrument having a second elongate body defining a first end and a second end, and a second handle portion at the first end, wherein the second end of the second elongate body is releasably engageable with the prosthesis, and wherein the second elongate body defines an engagement structure slidably engageable with the track of the first instrument;
a third instrument having a third elongate body defining a first end and a second end, a third handle portion at the first end, and a cutting element having one or more cutting edges at the second end, wherein the third elongate body defines an engagement structure slidably engageable with the track of the first instrument; and
a fourth instrument having a fourth elongate body defining a first end and a second end, a fourth handle portion at the first end, and a prosthesis trial element at the second end, wherein the prosthesis trial element includes a portion sized and shaped substantially similar to the prosthesis, and wherein the fourth elongate body defines an engagement structure slidably engageable with the track of the first instrument.

24. A method of implanting a prosthesis, comprising the steps of:
positioning a first tool at least partially between a first vertebral body and a second vertebral body;
slidably engaging a second tool to the first tool, wherein the second tool includes a first tip element; and
positioning the tip element between the first vertebral body and the second vertebral body.

25. The method according to claim 24, further comprising the step of making a cut in at least one of the first and second vertebral bodies with the tip element of the second tool.

26. The method according to claim 24, further comprising the steps of disengaging the first tip element from the second tool and releasably engaging a second tip element to the second tool.

27. The method according to claim 24, further comprising the steps of:
positioning a third tool at least partially between the first vertebral body and the second vertebral body; and
releasably engaging the third tool to the first tool such that the third tool and the first tool are substantially parallel.

28. The method according to claim 27, further comprising the steps of:
disengaging the second tool from the first tool; and
slidably engaging the second tool to the third tool; and
positioning the tip element between the first vertebral body and the second vertebral body.

29. The method according to claim 24, further comprising the steps of:
disengaging the second tool from the first tool;
slidably engaging a third tool to the first tool, wherein the third tool is releasably engaged to the prosthesis; and
positioning the prosthesis between the first vertebral body and the second vertebral body.
